(19) United States
(12) Patent Application Publication
(10) Pub. No.: US 2016/0106928 A1
(43) Pub. Date: Apr. 21, 2016

(54) MULTI-STAGE MIXING SYRINGE

(71) Applicant: NEOMED, INC., Woodstock, GA (US)

(72) Inventors: Benjamin Martin DAVIS, Woodstock, GA (US); Mariann WOLVERTON, Canton, GA (US); Elizabeth MURAYA, Woodstock, GA (US); Aaron N. INGRAM, Canton, GA (US)

(73) Assignee: NEOMED, INC., Woodstock, GA (US)

(21) Appl. No.: 14/872,237

(22) Filed: Oct. 1, 2015

Related U.S. Application Data
(60) Provisional application No. 62/064,590, filed on Oct. 16, 2014.
(60) Provisional application No. 62/192,454, filed on Jul. 14, 2015.
(60) Provisional application No. 62/207,120, filed on Aug. 19, 2015.

(51) Int. Cl. A61M 5/315; A61J 15/00; A61M 5/14

(52) U.S. Cl. CPC A61M 5/31596; A61M 5/1409; A61J 15/0026; A61M 2202/0482

(57) ABSTRACT

A system and method for mixing fluid constituents within a multi-stage mixing syringe, and delivering the mixture formed in the mixing syringe. The multi-stage mixing syringe system includes an outer syringe barrel defining a first contained volume therein, an intermediate syringe barrel and plunger configured for coupling within the outer syringe barrel and for advancement and retraction within the first contained volume, and defining a second contained volume therein, and an inner syringe plunger configured for coupling within the intermediate syringe barrel and plunger and for advancement and retraction within the second contained volume.

### CROSS-REFERENCE TO RELATED APPLICATIONS

This application claims the benefit of U.S. Provisional Patent Application Ser. No. 62/064,590 filed Oct. 16, 2014, U.S. Provisional Patent Application Ser. No. 62/192,454 filed Jul. 14, 2015, and U.S. Provisional Patent Application Ser. No. 62/207,120 filed Aug. 19, 2015, all of which are hereby incorporated herein by reference for all purposes.

### TECHNICAL FIELD

The present invention relates generally to the field of medical devices for enteral fluid delivery, and more particularly to a multi-stage mixing syringe for preparation and delivery of fluids such as formula, breast milk, enteral supplements and medications.

### BACKGROUND

Syringes are often used in delivery of fluids for medical applications. For example, a syringe can be utilized for preparation, storage and enteral delivery of fluids to a neonate or other human or animal recipient through a feeding tube, nipple or other delivery means. Typically, a single type of enteral fluid material such as breast milk or formula is administered from a standard syringe having a barrel and a plunger slidably mounted in the barrel. Advancing the plunger into the barrel, either manually or using an automated syringe pump, pressurizes the fluid within the barrel and discharges the fluid from a nipple or nozzle outlet of the barrel.

In some instances, it has been found desirable to mix multiple fluids together prior to administration. For example a nutritional supplement or medication may be more readily administered to a neonate along with formula or breast milk. Typically this is accomplished by mixing the fluid in a mixing container, and then filling the syringe with the desired quantity of fluid from the mixing container. Previously known systems and methods of mixing and delivering such fluids may expose the fluid to air or potential contaminants, increase the risk of spilling or waste, and/or require substantial preparation time and equipment.

Additionally, many known syringe designs used for enteral fluid delivery can be coupled to non-enteral equipment, creating potential risks of inadvertent enteral delivery of non-enteral fluids, and/or are not compatible with the ENFit design standard (ISO 80369-3) for enteral fluid connections.

Accordingly, it can be seen that needs exist for improved systems and methods for preparation, storage and delivery of fluids. It is to the provision of improved systems and methods meeting these and other needs that the present invention is primarily directed.

### SUMMARY

In example embodiments, the present invention provides improved systems and methods for preparation, storage and delivery of fluids. In example applications, the present invention provides a multi-stage mixing syringe wherein two or more fluid components may be mixed in a closed and/or aseptic containment volume prior to delivery, and delivered from the syringe to a human or animal recipient, for example for enteral fluid delivery applications, without the need to transfer the fluid to the syringe from a mixing container.

In one aspect, the present invention relates to a multi-stage mixing syringe system. The system preferably includes an outer syringe barrel defining a first contained volume therein; an intermediate syringe barrel and plunger configured for coupling within the outer syringe barrel and for advancement and retraction within the first contained volume, and defining a second contained volume therein; and an inner syringe plunger configured for coupling within the intermediate syringe barrel and plunger and for advancement and retraction within the second contained volume.

In another aspect, the invention relates to a method of mixing first and second constituent fluids and delivering a mixture thereof. The method preferably includes at least partially filling a first syringe stage of a multi-stage mixing syringe with the first constituent fluid, at least partially filling a second stage of the multi-stage mixing syringe with the second constituent fluid, actuating the first syringe stage of the multi-stage mixing syringe to mix the first and second constituent fluids within the multi-stage mixing syringe, and forming the mixture of the first and second constituent fluids within the multi-stage mixing syringe. Optionally, the method further includes the step of actuating the second syringe stage of the multi-stage mixing syringe to discharge the mixture therefrom.

In example forms, the multi-stage mixing syringe system and the method of mixing first and second constituent fluids and delivering a mixture thereof are particularly adapted to enteral fluid delivery. For example, the multi-stage mixing syringe comprises an enteral fluid delivery syringe configured to mix a first constituent for enteral delivery with a second constituent for enteral delivery, thereby forming a substantially homogenous enteral fluid mixture, and to deliver the enteral fluid mixture to a human or animal subject.

In further aspects, the invention relates to a multi-stage enteral mixing syringe including an outer syringe barrel defining a first contained volume therein; an intermediate syringe barrel configured for coupling within the outer syringe barrel and for advancement and retraction within the first contained volume, and defining a second contained volume therein; and an inner syringe plunger configured for coupling within the intermediate syringe barrel and plunger and for advancement and retraction within the second contained volume. The outer syringe barrel includes an enteral coupling, for example an enteral-only coupling such as an ENFit design standard (ISO 80369-3) compatible coupling, optionally including a low-dose tip configuration.

In another aspect, the invention relates to a multi-stage mixing syringe system including an outer syringe barrel defining a first contained volume therein for receiving a first enteral fluid constituent, and an enteral fluid delivery port at a distal end of the outer syringe barrel in fluid communication with the first contained volume. The multi-stage mixing syringe system preferably further includes an intermediate syringe barrel and plunger configured for coupling within the outer syringe barrel and for advancement and retraction within the first contained volume, and defining a second contained volume therein for receiving a second enteral fluid constituent. The multi-stage mixing syringe system preferably also includes an inner syringe plunger configured for coupling within the intermediate syringe barrel and plunger and for advancement and retraction within the second contained volume.

In another aspect, the invention relates to a multi-stage enteral syringe for mixing and delivering enteral fluid. The multi-stage enteral syringe preferably includes an outer syringe barrel defining a first internal chamber and comprising an enteral fluid delivery port in fluid communication with the first internal chamber. The multi-stage enteral syringe preferably also includes an intermediate syringe barrel and plunger received in sealing engagement within the first internal chamber of the outer syringe barrel, the intermediate syringe barrel and plunger defining a second internal chamber and comprising a fluid transfer port providing fluid communication between the first and second internal chambers. The multi-stage enteral syringe preferably also includes an inner plunger received in sealing engagement within the second internal chamber of the intermediate syringe barrel and plunger. The multi-stage enteral syringe preferably also includes an enteral fluid constituent material preloaded within the second internal chamber of the intermediate syringe barrel and plunger.

In another aspect, the invention relates to a method of mixing first and second constituent materials to form an enteral fluid mixture and delivering the enteral fluid mixture to a human or animal subject. The method preferably includes at least partially filling a first syringe stage of a multi-stage mixing syringe with the first constituent material, at least partially filling a second stage of the multi-stage mixing syringe with the second constituent material, actuating the first syringe stage of the multi-stage mixing syringe to mix the first and second constituent materials together to form the enteral fluid mixture, and actuating the second stage of the multi-stage mixing syringe to deliver the enteral fluid mixture to the gastrointestinal system of a human or animal subject.

These and other aspects, features and advantages of the invention will be understood with reference to the drawing figures and detailed description herein, and will be realized by means of the various elements and combinations particularly pointed out in the appended claims. It is to be understood that both the foregoing general description and the following brief description of the drawings and detailed description of the invention are exemplary and explanatory of preferred embodiments of the invention, and are not restrictive of the invention, as claimed.

### BRIEF DESCRIPTION OF THE DRAWINGS

FIG. 1 shows a multi-stage mixing syringe system according to an example embodiment of the present invention, in a first configuration.

FIG. 2 shows the multi-stage mixing syringe system of FIG. 1 in a second configuration.

FIG. 3 shows the multi-stage mixing syringe system of FIG. 1 in a third configuration.

FIG. 4 shows the multi-stage mixing syringe system of FIG. 1 in a fourth configuration.

FIG. 5 shows a multi-stage mixing syringe system according to another example embodiment of the present invention.

### DETAILED DESCRIPTION OF EXAMPLE EMBODIMENTS

The present invention may be understood more readily by reference to the following detailed description of the invention taken in connection with the accompanying drawing figures, which form a part of this disclosure. It is to be understood that this invention is not limited to the specific devices, methods, conditions or parameters described and/or shown herein, and that the terminology used herein is for the purpose of describing particular embodiments by way of example only and is not intended to be limiting of the claimed invention. Any and all patents and other publications identified in this specification are incorporated by reference as though fully set forth herein.

Also, as used in the specification including the appended claims, the singular forms “a,” “an,” and “the” include the plural, and reference to a particular numerical value includes at least that particular value, unless the context clearly dictates otherwise. Ranges may be expressed herein as from “about” or “approximately” one particular value and/or to “about” or “approximately” another particular value. When such a range is expressed, another embodiment includes from the one particular value and/or to the other particular value. Similarly, when values are expressed as approximations, by use of the antecedent “about,” it will be understood that the particular value forms another embodiment.

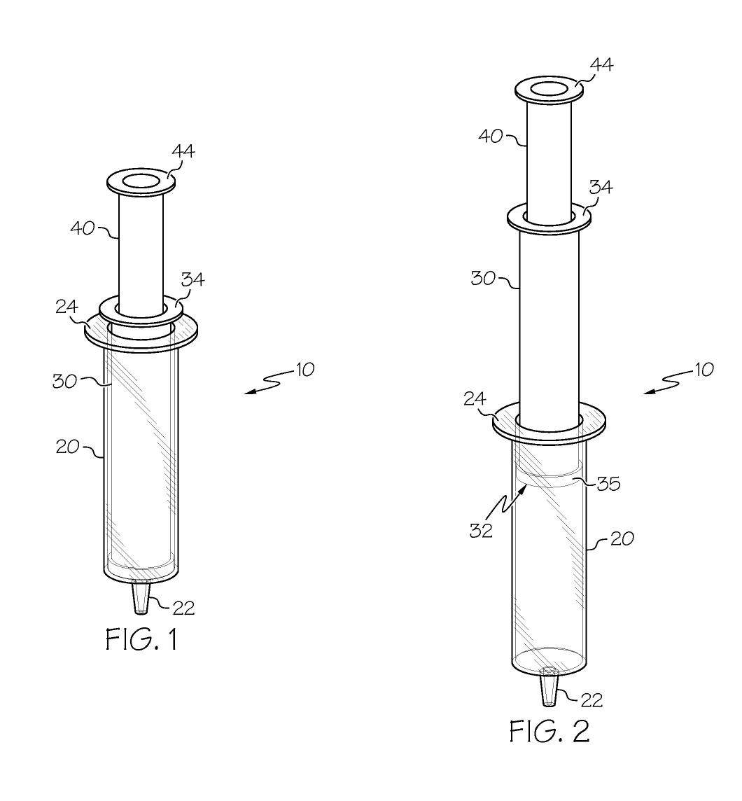

With reference now to the drawing figures, wherein like reference numbers represent corresponding parts throughout the several views, FIGS. 1-4 show a multi-stage mixing syringe system 10 according to an example embodiment of the present invention, through a sequence of configurations demonstrating an example method of use in the preparation, storage and delivery of a fluid comprising a mixture of at least two fluid constituents.

The multi-stage mixing syringe system 10 generally comprises an outer syringe barrel 20, an intermediate syringe barrel and plunger 30, and an inner syringe plunger 40. In example form, the outer syringe barrel 20 comprises an axially elongate, generally cylindrical hollow body or shell, with a nipple or fluid delivery port 22 at its distal end and a transversely extending flange 24 at its proximal end. In alternate embodiments, the body of the outer syringe barrel 20 defines a circular, oval, teardrop-shaped, rectangular, polygonal or otherwise shaped cross-sectional profile. Thus, the body of the outer syringe barrel 20 defines a first internal contained volume or containment chamber having a lengthwise or axial dimension (vertical in the drawing figures) extending at least partially between the proximal and distal ends of the outer syringe barrel, and an inside diameter or transverse dimension (horizontal in the drawing figures) for containing a first quantity of a first constituent fluid or material therein. In example form, the fluid delivery port or nipple 22 defines an internal lumen or conduit comprising a smaller-dimensioned distal opening or fluid delivery port, and a larger-dimensioned proximal opening generally coincident with the internal surface of the contained volume within the body of the outer syringe barrel 20. Optionally, the nipple 22 comprises an enteral coupling such as an ENFit or other enteral only coupling, a PGLock, a Luer coupling, or another specialized or standard coupling element. The body of the outer syringe barrel 20 may be constructed of a generally transparent or translucent material, and a gauge or scale may be marked or otherwise formed with incremental volumetric (e.g., mL or cc) markings spaced axially along the body of the outer syringe barrel, whereby the volume of contents within the body of the outer syringe barrel can be visually determined.

The intermediate syringe barrel and plunger 30 also comprises an axially elongate, generally cylindrical hollow body or shell, with a distal end 32 defining an opening or transfer port therethrough, and a transversely extending flange 34 at its proximal end. The body of the intermediate syringe barrel and plunger 30 comprises an external geometry generally corresponding to the internal geometry of the outer syringe barrel 20, whereby the intermediate syringe barrel and plunger can slide axially within the outer syringe barrel. The external geometry of the body of the intermediate syringe barrel and plunger 30 preferably has a length in the axial direction (vertical in the drawing figures) equal to or slightly greater than the length of the first internal contained volume within the outer syringe barrel 20, and an outer diameter or transverse dimension (horizontal in the drawing figures) equal to or slightly smaller than the inside diameter or transverse dimension of the first internal contained volume within the outer syringe barrel. One or more seals 35 such as sealing rings or gaskets can be provided on the external surface of the distal end 32 of the intermediate syringe barrel and plunger 30 for sealing and sliding engagement with the internal surface of the outer syringe barrel 20, or a closely-toleranced fit can be provided between the exterior of the intermediate syringe barrel and plunger and the interior of the outer syringe barrel, to maintain a fluid-tight sealed contact therebetween. The size and configuration of the transfer port or opening through the distal end 32 of the intermediate syringe barrel and plunger 30 may vary depending upon the constituent fluid to be delivered. For example, for a low-viscosity liquid constituent fluid, a smaller transfer port may be utilized to deliver a higher velocity flow from the intermediate syringe barrel and plunger 30 into the outer syringe barrel 20 for better mixing. Alternatively, for a higher viscosity liquid, gel, powder or particulate constituent fluid, a larger transfer port may be utilized to deliver a larger flow volume.

The body of the intermediate syringe barrel and plunger 30 comprises an internal geometry defining a second internal contained volume or containment chamber having a lengthwise or axial dimension (vertical in the drawing figures) extending at least partly between the proximal and distal ends of the intermediate syringe barrel and plunger, and an inside diameter or transverse dimension (horizontal in the drawing figures) for containing a second quantity of a second constituent fluid or material therein. Thus, when the intermediate syringe barrel and plunger 30 is installed within the outer syringe barrel 20, the opening or fluid transfer port at the distal end 32 of the intermediate syringe barrel and plunger defines a fluid conduit between the first and second internal contained volumes of the system. Optionally, the fluid transfer port or opening through the distal end 32 of the intermediate syringe barrel and plunger 30 may be sealed by a releasable seal such as a pressure releasable or frangible flap or closure to prevent fluid transfer into and/or out of the intermediate syringe barrel until a threshold pressure or force is applied, and to allow fluid transfer when the threshold pressure or force is reached.

The inner syringe plunger 40 comprises an elongate body having a distal end configured for sliding and sealing engagement within the second internal contained volume in the intermediate syringe barrel and plunger 30, and a proximal end comprising a transverse flange 44. The inner syringe plunger 40 has a length in the axial direction (vertical in the drawing figures) equal to or slightly greater than the length of the second internal contained volume within the intermediate syringe barrel and plunger 30, and an outer diameter or transverse dimension (horizontal in the drawing figures) equal to or slightly smaller than the inside diameter or transverse dimension of the second internal contained volume within the intermediate syringe barrel and plunger. One or more seals such as sealing rings or gaskets can be provided at the exterior of the distal end of the inner syringe plunger 40 for sealing and sliding engagement with the interior surface of the intermediate syringe barrel and plunger 30, or a closely-toleranced fit can be provided, to maintain a fluid-tight sealed contact therebetween.

Thus, when assembled, the system 10 comprises the inner syringe plunger 40 inserted within the intermediate syringe barrel and plunger 30, and the intermediate syringe barrel and plunger inserted within the outer syringe barrel 20. In this manner, an inner syringe stage of the system 10 is defined by the inner syringe plunger 40 (which functions as the plunger of the first syringe stage) advancing and/or retracting within the intermediate syringe barrel and plunger 30 (which functions as the barrel of the first syringe stage); and an outer syringe stage of the system is defined by the intermediate syringe barrel and plunger 30 (which functions as the plunger of the second syringe stage) advancing and/or retracting within the outer syringe barrel 20 (which functions as the barrel of the second syringe stage).

In further example embodiments, the multi-stage mixing syringe system can comprise two, three, four or more syringe stages, allowing for mixture of two, three, four or more constituent fluids. Optionally, a locking mechanism can be provided to lock the inner syringe plunger 40 into the intermediate syringe barrel 30 once the contents of the intermediate syringe barrel have been mixed into the outer syringe barrel.

In an example method of use of the system 10, illustrated sequentially in FIGS. 1-4, the inner syringe stage of the system 10 is prefilled with a quantity of one constituent fluid or material within the contained volume of the intermediate syringe barrel and plunger 30. In example forms, the constituent fluid or material of the inner stage can be a medication or nutritional supplement for enteral delivery in liquid, gel, powder or particulate form. The intermediate syringe barrel and plunger 30 is fully advanced into the outer syringe barrel 20, as shown in FIG. 1. If a releasable seal is provided at the opening or fluid transfer port at the distal end 32 of the intermediate syringe barrel and plunger, it is initially closed.

The outer syringe stage of the system is then loaded with a quantity of another constituent fluid or material by placing the fluid delivery port 22 in communication with a source of the fluid or material to be loaded, and retracting the intermediate syringe barrel and plunger 30 relative to the outer syringe barrel 20, as shown in FIG. 2. Alternatively, the outer syringe stage 20 of the system may also be prefilled, and a removable closure or cap installed on the fluid delivery port 22. In example forms, the constituent fluid or material of the inner stage can be an enteral fluid such as infant formula, breast milk, water or enteral nutrition product, for example in liquid, gel, powder or particulate form. Preferably the outer syringe stage of the system is filled to less than the capacity of the outer syringe barrel 20, allowing capacity for subsequent addition of the constituent fluid from the inner syringe stage. The fluid delivery port 22 may be capped to prevent discharge from the outer syringe stage.

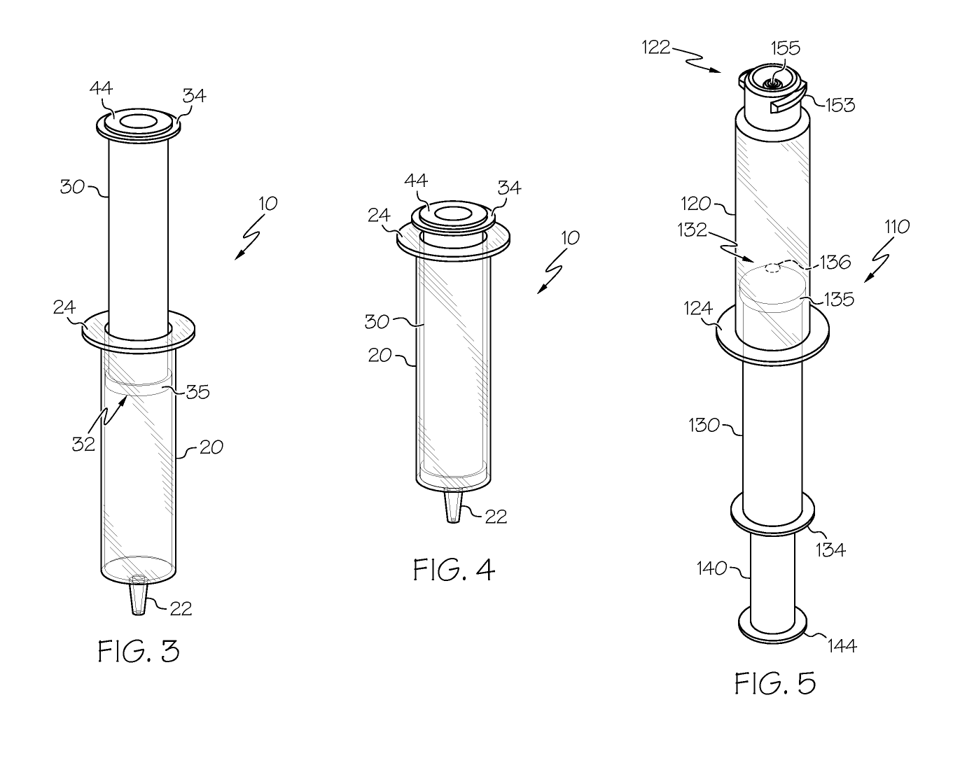

The inner syringe plunger 40 is then advanced into the intermediate syringe barrel and plunger 30 by pressing flange 44 toward flange 34, breaking or releasing the releasable seal (if present) at the discharge end 32 of the intermediate syringe barrel and plunger, and transferring the constituent fluid from the inner syringe stage through the fluid transfer port at the distal end of the intermediate syringe barrel from the contained volume within the intermediate syringe barrel and plunger to the contained volume within the outer syringe barrel 20, as shown in FIG. 3. The intermediate syringe barrel and plunger 30 retracts relative to the outer syringe barrel 20 as the inner syringe plunger is advanced, to increase the contained volume of the outer stage to accommodate the constituent fluid received from the inner syringe stage. The constituent fluids from the inner and outer stages are mixed within the closed containment of the syringe system, for example by shaking, to form a substantially homogenous mixture, without exposure to air or potential contaminants during the mixing process. Optionally, one or more stages of the syringe system may be sequentially advanced and retracted to enhance mixing of the constituent fluids. The fluid delivery port 22 may be de-capped, and the mixture delivered from the system by pressing the flange 34 toward the flange 24 and advancing the intermediate syringe barrel and plunger 30 within the outer syringe barrel 20.

The delivery of the mixture may be manually actuated, or alternatively may be carried out by use of an automated syringe pump or driver. In example applications, the mixture is an enteral feeding fluid delivered via a feeding tube having a first end coupled to the fluid delivery port 22 and a second end feeding the fluid to the gastrointestinal system of a neonate or other human or animal recipient. In alternate applications, the mixture may be an injectable medication delivered via a hypodermic needle coupled to the fluid delivery 22. In still further alternate forms, the multi-part mixing syringe system can be applied for uses other than medical. For example, the constituent fluids mixed by the syringe system to form a mixture may be any of a variety of fluids for medical or non-medical use, for example a sample-and-diluent used in laboratory applications, a two-part epoxy adhesive for industrial applications to attach workpieces or other parts, a fuel-and-additive mixture, a topical medication, a paint and hardening agent, etc. For medical applications, the syringe system may optionally be sterilized and aseptically packaged, whereas for industrial or other non-medical applications the syringe system may be non-sterile and packaged in bulk.

In alternate applications, the multi-stage syringe system may be utilized for purposes other than mixing of multiple constituent fluids. For example, the same fluid may be loaded in both the inner syringe stage and the outer syringe stage, and a first actuation of one of the stages deliver a first dose or quantity of the fluid and a second actuation of the other stage deliver a second dose or quantity of the fluid. Alternatively, the fluid to be delivered may be contained in only one of the syringe stages, and the other syringe stage used as a delivery vehicle, for protective containment, temperature control, flushing or priming a delivery conduit, or other uses.

FIG. 5 shows another example embodiment of a multi-stage syringe system 110 according to the invention, having substantially similar structure and manner of use to the above-described system, except as noted. The outer stage of the syringe system 110 comprises an outer syringe barrel 120 having a distal end 122 and a transversely extending proximal end flange 124, and an intermediate syringe barrel and plunger 130 for advancement and retraction relative to the outer syringe barrel. The inner stage of the syringe system 110 comprises the intermediate syringe barrel and plunger 130, and an inner plunger 140. The intermediate syringe barrel and plunger 130 has a distal end 132 with sealing head 135 and an enteral fluid transfer port 136 for fluid transfer between the inner contained volume of the intermediate syringe barrel and the outer contained volume of the outer syringe barrel, and a proximal end comprising a transversely extending flange 134. The inner plunger 140 comprises a sealing head at its distal end and a transversely extending flange 144 at its proximal end. The outer syringe barrel 120 comprises an enteral only fluid delivery port at its distal end 122, in the form of an ENFit design standard (ISO 80369-3) compatible coupling with external thread elements 153 and an inner coaxial low-dose tip 155 having a small diameter lumen extending therethrough in fluid communication with the contained volume of the outer syringe barrel 120.

While the invention has been described with reference to preferred and example embodiments, it will be understood by those skilled in the art that a variety of modifications, additions and deletions are within the scope of the invention, as defined by the following claims.

What is claimed is:

1. A multi-stage mixing syringe system comprising:
an outer syringe barrel defining a first contained volume therein for receiving a first enteral fluid constituent, and an enteral fluid delivery port at a distal end of the outer syringe barrel in fluid communication with the first contained volume;
an intermediate syringe barrel and plunger configured for coupling within the outer syringe barrel and for advancement and retraction within the first contained volume, and defining a second contained volume therein for receiving a second enteral fluid constituent; and
an inner syringe plunger configured for coupling within the intermediate syringe barrel and plunger and for advancement and retraction within the second contained volume.

2. The multi-stage mixing syringe system of claim 1, wherein the intermediate syringe barrel and plunger comprises a transfer port providing fluid communication between the first and second contained volumes.

3. The multi-stage mixing syringe system of claim 2, further comprising a releasable seal preventing fluid passage through the transfer port in a closed state and allowing fluid passage through the port in an open state.

4. The multi-stage mixing syringe system of claim 1, wherein the enteral fluid delivery port comprises an ENFit design standard compatible coupling.

5. The multi-stage mixing syringe system of claim 4, wherein the enteral fluid delivery port further comprises a low-dose tip.

6. The multi-stage mixing syringe system of claim 1, wherein the outer syringe barrel is generally transparent or translucent.

7. The multi-stage mixing syringe system of claim 1, wherein the second contained volume is at least partially prefilled with the second enteral fluid constituent.

8. The multi-stage mixing syringe system of claim 7, wherein the second enteral fluid constituent is selected from a medication, a nutritional supplement, or a combination thereof.

9. The multi-stage mixing syringe system of claim 1, wherein the first enteral fluid constituent is selected from infant formula, breast milk, water, an enteral nutrition product, or a combination thereof.

10. The multi-stage mixing syringe system of claim 1, further comprising a feeding tube having a coupling for cooperative engagement with the enteral fluid delivery port.

11. The multi-stage mixing syringe system of claim 1, further comprising an automated syringe pump.

12. A multi-stage enteral syringe for mixing and delivering enteral fluid comprising:
an outer syringe barrel defining a first internal chamber and comprising an enteral fluid delivery port in fluid communication with the first internal chamber;
an intermediate syringe barrel and plunger received in sealing engagement within the first internal chamber of the outer syringe barrel, the intermediate syringe barrel and plunger defining a second internal chamber and comprising a fluid transfer port providing fluid communication between the first and second internal chambers;
an inner plunger received in sealing engagement within the second internal chamber of the intermediate syringe barrel and plunger; and
an enteral fluid constituent material preloaded within the second internal chamber of the intermediate syringe barrel and plunger.

13. The multi-stage enteral syringe of claim 12, wherein the enteral fluid delivery port of the outer syringe barrel comprises an enteral only coupling.

14. The multi-stage enteral syringe of claim 12, wherein the enteral fluid delivery port of the outer syringe barrel comprises an ENFit design standard compatible coupling.

15. The multi-stage enteral syringe of claim 13, wherein the ENFit design standard compatible coupling further comprises a low-dose tip.

16. The multi-stage enteral syringe of claim 12, wherein the enteral fluid constituent material preloaded within the second internal chamber of the intermediate syringe barrel and plunger is selected from a medication, a nutritional supplement, or a combination thereof.

17. A method of mixing first and second constituent materials to form an enteral fluid mixture and delivering the enteral fluid mixture to a human or animal subject, the method comprising:
at least partially filling a first syringe stage of a multi-stage mixing syringe with the first constituent material;
at least partially filling a second stage of the multi-stage mixing syringe with the second constituent material;
actuating the first syringe stage of the multi-stage mixing syringe to mix the first and second constituent materials together to form the enteral fluid mixture; and
actuating the second stage of the multi-stage mixing syringe to deliver the enteral fluid mixture to the gastrointestinal system of a human or animal subject.

18. The method of claim 17, wherein the step of actuating the second syringe stage of the multi-stage mixing syringe to discharge the mixture therefrom is carried out by an automated syringe pump.
